(19) United States
(12) Patent Application Publication
(10) Pub. No.: US 2004/0076690 A1
(43) Pub. Date: Apr. 22, 2004

(54) Compositions for weight loss with improved taste

(75) Inventors: Takeshi Ikemoto, Minamiashigara-Shi (JP); Hiroyuki Nishio, Fujisawa-Shi (JP); Mariko Hara, Odawara-Shi (JP); Masato Saito, Odawara-Shi (JP); Satoshi Yomoda, Kobe-Shi (JP); Koichi Ogata, Ibaraki-Shi (JP); Kenya Ishida, Yokohama-shi (JP); Tamami Sato, Chigasaki-shi (JP); Hiromi Matsumoto, Tokyo (JP); Toshiyuki Hamasaki, Saitama-shi (JP); Yoshiko Nihei, Kawasaki-Shi (JP); Naoko Ozawa, Odawara-Shi (JP); Charles H. Manley, Ringwood, NJ (US)

Correspondence Address: DARBY & DARBY P.C., Post Office Box 5257, New York, NY, 10150-5257, US

(73) Assignees: Kanebo, Ltd., Tokyo (JP); Takasago International Corporation, Tokyo (JP); Takasago International Corporation, Rockleigh, NJ

(21) Appl. No.: 10/426,939

(22) Filed: Apr. 30, 2003

(30) Foreign Application Priority Data
Apr. 30, 2002 (JP) 2002-128770
May 30, 2002 (JP) 2002-156911

(51) Int. Cl. A61K 31/70; A61K 31/12; A61K 31/075; A61K 35/78; A01N 31/14

(52) U.S. Cl. 424/729; 424/736; 424/756; 424/757; 424/764; 424/750; 514/25; 514/678; 514/718

(57) ABSTRACT

The present invention relates to compositions comprising compounds that induce weight and fat loss and compounds that mask the bitter and harsh taste of the composition. The invention further relates to a food, beverage, dietary supplement, medical food, or pet food comprising compounds that induce weight loss and compounds that mask the bitter and harsh taste of the product. The invention is further related to methods of reducing or controlling weight by administering such compositions. Further provided are cosmetic sheets for topical administration of compositions for inducing weight and fat loss.

This application claims the benefit of Japanese Patent Application No. 2002-128770, filed Apr. 30, 2002, and Japanese Patent Application No. 2002-156911, filed May 30, 2002, both of which are hereby incorporated by reference.

### FIELD OF THE INVENTION

The present invention relates to compounds that induce weight loss and fat loss and compositions and clathrates that mask the bitter and harsh taste of such compounds. The invention further relates to a food, beverage, dietary supplement, medical food, pet food, cosmetic or bath salt comprising compounds that induce weight loss and compositions and clathrates that mask the bitter and harsh taste of,the product. The invention is further related to methods of reducing or controlling weight or fat by administering such compositions or clathrates.

### BACKGROUND OF THE INVENTION

In developed countries, obesity has increased in recent years due to overall lifestyle changes including increased caloric intake and insufficient exercise. In turn, being overweight or obese is directly or indirectly associated with a vast number of diseases including high blood pressure, diabetes, heart disease, and gallstones. People who are overweight often experience more health problems and shortened life expectancies. Therefore, there continues to be a search for new and effective means to facilitate weight and fat loss or to control weight and fat.

Herbal and natural products that contain gymunema extract, garcinia extract, or carnitine are known to prevent fat accumulation through the inhibition of fat absorption, enhancement of fat decomposition, and the enhancement of fat consumption by the body. The active ingredients in these extracts, however, have a characteristic bitter and harsh taste and peculiar aroma. For example, hydroxy citric acid (HCA), an active component in garcinia, is known to inhibit the citric acid ATP lipase that metabolizes sugar to fat. HCA, however, has a characteristic bitter and harsh taste, and the addition of an amount of HCA effective to cause weight or fat loss spoils food flavor.

Hydroxyphenylbutan-2-ones or hydroxyphenylbutan-2-ols of formulas (1) and (2), represented by the formula

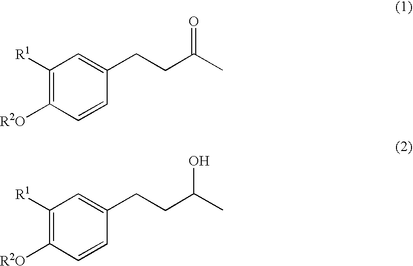

where

R¹=H, —OH, or —OCH₃

R²=H, C—OR³, or sugar group and

R³=C₂-C₂₀ alkyl,

have been found to enhance fat decomposition (JP 2000-169325 and JP 2001-226263). The compounds raspberry ketone (4-(4-hydroxyphenyl)-butan-2-one) and zingerone (4-(3-methoxy-4-hydroxyphenyl)-butan-2-one) and BK-180 (4-(3,4-dihydroxyphenyl)butan-2-one) and its derivatives, for example, have the ability to promote the decomposition of fats accumulated in fatty tissue and are effective for inhibiting obesity and for improving pyknic constitutions (Japanese Patent Laid-Open No. 2000-169325).

Compounds of formulas (1) and (2) are less pungent and bitter, compared to other natural weight loss ingredients, such as capsaicin. However, compounds of formulas (1) and (2), e.g., raspberry ketone and zingerone, have a taste and peculiar aroma that are unpleasant when these compounds are present in high amounts. Despite these complications, hydroxyphenylbutan-2-ones or hydroxyphenylbutan-2-ols of formulas (1) and (2) are used satisfactorily as minor substituents and relatively low amounts in compositions to impart fragrance or flavor. Large amounts of these compounds are necessary, however, to achieve weight loss. Accordingly, they are generally unsatisfactory for use in compositions (e.g., food, tablets, drink, pet food, cosmetics or bath salts) designed to effect loss of weight or fat, due to the unpleasant aroma and taste associated with the large amounts of the compounds.

In order to mask the unpleasant taste of these active ingredients, components such as sweeteners or particular flavors can be added to compositions. These additives, however, do not always effectively mask active ingredients that have a strong bitter taste. Nor are they typically effective in masking unpleasant aromas. There is, therefore, a need for improved methods of masking unpleasant taste and/or aromas of active ingredients of formulas (1) and (2).

It has now been found that the unpleasant taste and aroma of compounds of formula (1) and formula (2) may be masked with specific flavorants, through encapsulation methods, or by including compounds of formula (1) or (2) in a clathrate. The ability to mask the unpleasant taste and/or aroma of compounds of formulas (1) and (2) permits application or ingestion of higher amounts of these compounds by a mammal, directly or in a composition, leading to increased efficacy of the compounds in fat reduction and weight loss.

### SUMMARY OF THE INVENTION

The invention is directed to compositions comprising (i) one or more compound represented by the formulas (1) and/or (2)

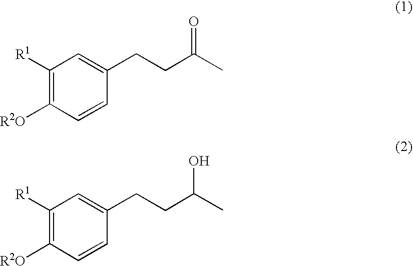

wherein R¹=H, —OH, or —OCH₃, R²=H, C—OR³, or sugar group and R³=C₂-C₂₀ alkyl, and (ii) one or more compound that masks or lessens an unpleasant taste or aroma of a compound of formula (1) or formula (2).

Accordingly, in one aspect, the invention is drawn to a composition comprising: (i) one or both of a compound of formula (1) and a compound of formula (2) and (ii) an effective taste masking amount of one or more member selected from the group consisting of extracts of oolong tea, puerh tea, roasted adlay tea, eucommia leaves, jasmine flower, banzakuro (guava fruit), barley, mate tea (Paraguay tea), rooibos tea, tinpi (matured peal of Citrus unshiu), juemingzi, sanzashi (Crataegus), soybean germ, ginger, ginseng, kouki tea, and yucca. Preferably, the member is selected from the group consisting of extracts from oolong tea, puerh tea, roasted adlay tea, eucommia leaves, and jasmine flower.

In another aspect, the invention is drawn to a composition comprising: (i) one or both of a compound of formula (1) and a compound of formula (2) and (ii) an effective taste-masking amount of one or more members selected from the group consisting of malthol, ethyl maltol, furaneol, furaneol derivatives, vanillin, and ethyl vanillin.

In another aspect, the invention is drawn to a clathrate comprising a compound of formula (1) or a compound of formula (2). In a preferred embodiment, the clathrate comprises a compound of formula (1) or formula (2) included in a cyclodextrin.

In further aspects, the foregoing compositions or clathrates are suitable for administration to a mammal, and most preferably to a human, dog, or cat.

In further aspects, the invention is also directed to methods of using the foregoing compositions or clathrates to effect weight loss and/or fat reduction in a mammal, and preferably a human, dog or cat.

In preferred embodiments, any of the foregoing compositions or clathrates are added to, mixed with, or formulated as, a food, beverage, dietary supplement, medical food, pet food, cosmetic, topical application sheet, or bath salt.

In certain embodiments, the invention is drawn to a method of reducing or controlling weight, e.g., increasing weight loss, or reducing or controlling fat by administering a clathrate including compound of formula (1) or (2) or a composition comprising one or both of a compound of formula (1) and a compound of formula (2) in a formulation that masks an unpleasant taste or odor of the compound. In preferred embodiments, the administered composition is a composition as set forth above. Most preferably, the clathrate or composition is administered in or as a food, beverage, dietary supplement, medical food, pet food, cosmetic, or bath salt comprising a clathrate or composition as set forth above. The clathrate or composition is preferably administered to a mammal. Most preferably, the clathrate or composition is administered to a human, dog, or cat.

### BRIEF DESCRIPTION OF THE DRAWINGS

FIG. 1 is a differential scanning calorimetry pattern of raspberry ketone heated at 10° C./min.

FIG. 2 is a differential scanning calorimetry pattern of β-CD heated at 10° C./min.

FIG. 3 is a differential scanning calorimetry pattern of a mixture of β-CD and raspberry ketone (9:1 by weight) heated at 10° C./min.

FIG. 4 is a differential scanning calorimetry pattern of the clathrate of Example 7 heated at 10° C./min.

FIG. 5 is a ¹H-NMR spectral pattern of raspberry ketone.

FIG. 6 is a ¹H-NMR spectral pattern of the clathrate of Example 7.

### DETAILED DESCRIPTION OF THE INVENTION

Compounds of formulas (1) and (2) have the property of reducing or controlling weight and/or reducing or controlling fat when administered to a mammal. Compounds of formulas (1) and (2) have certain undesirable properties, however, that, in the past, have limited their use in compositions and products that are effective in controlling and reducing weight and fat. These undesirable properties include low solubility in water and unpleasant taste and aroma, when compounds of formula (1) or (2) are present in large amounts. As described herein, the present inventors now provide new and improved compounds and compositions that overcome undesirable taste and aroma characteristics of compounds of formulas (1) and (2).

As described herein, the invention provides compositions that include amounts of compounds of formula (1) and/or (2) sufficient to effect weight and fat loss, in which the unpleasant taste and aroma associated with compounds of formula (1) and/or (2) has been masked. Also provided are new and improved compounds in which compounds of formula (1) and/or (2) have been included in a clathrate. The clathrates increase the solubility of compounds of formula (1) and (2) and mask their unpleasant taste and aroma. The new and improved compositions and clathrates are useful for administrating to a mammal (e.g., a human, dog or cat) to effect weight or fat loss.

In particular, in regard to certain aspects of the invention, the present inventors have found that, when hydroxyphenylbutan-2-ones or hydroxyphenylbutan-2-ols having an effect of fat decomposition and weight reduction are included in cyclodextrin (“CD”), the solubility of the compounds in water, food, beverage, dietary supplement, medical food, pet food, cosmetic, or others is increased. The unpleasant taste and aroma characteristics of the compounds are also reduced, without detracting from the effectiveness of the compounds for fat decomposition, body reduction, and weight loss.

Hence, in certain embodiments, the invention improves the solubility in water of hydroxyphenylbutan-2-ones and hydroxyphenylbutan-2-ols of formula (1) or (2) and improves the taste and aroma properties of the compounds. Specifically, the invention also provides a composition of that compound, which is highly soluble in water, food, beverage, dietary supplement, medical food, pet food, cosmetic, or the like and has the advantages of pleasant taste and aroma, and stability, and which is effective for promoting fat decomposition, body reduction, and weight reduction.

Compounds of Formulas (1) and (2)

Compounds of formulas (1) and (2) have the following structures:

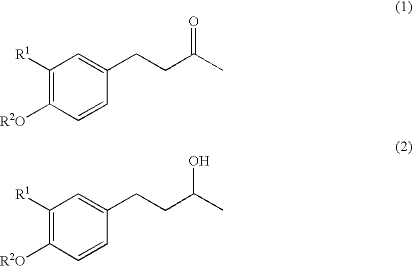

wherein

R¹=H, —OH, or —OCH₃,

R²=H, —COR³, or sugar group, and

R³=C₂-C₂₀ alkyl,

Examples of sugar groups that may be R² include, but are not limited to, monosaccharides such as glucose, galactose, mannose, rhamnose, xylose, ribose, arabinose, glucosamine, and galactosamine, and disaccharides such as lactose, maltose, cellobiose, isomaltose, and epilactose.

Specific examples of compounds of formula (1) include compounds contained in the fruit raspberry. For example, in raspberry ketone, R¹ and R² are H and in zingerone (4-(3-methoxy-4-hydroxyphenyl)-butane-2-one), R¹ is OCH₃, R² is H. Raspberry ketone and zingerone both have a characteristic bitter and harsh taste and an unpleasant aroma, when present in large amounts.

Other examples of compounds of formula (1) include, without limitation, 4-(3,4-dihydroxyphenyl)-butane-2-one (formula (1) in which R¹ is OH and R² is H) and glycosides, including raspberry ketone glucoside (formula (1) in which R¹ is H and R² is glucose) and raspberry ketone galactoside (formula (1) in which R¹ is H and R² is galactose).

Examples of compounds of formula (2) include, without limitation, 4-(4-hydroxyphenyl)-butane-2-ol (formula (2) in which R¹ and R² are H), 4-(3,4,-dihydroxyphenyl)butane-2-ol (formula (2) in which R¹ is OH and R² is H), 4-(3-methoxy-4-hydroxyphenyl)-butane-2-ol (formula (2) in which R¹ is OCH₃ and R² is H), and glycosides, for example glucoside and galactoside, thereof.

Hydroxyphenylbutan-2-ones for use in the invention include, for example and without limitation, 4-(4-hydroxyphenyl)butan-2-one, 4-(4-hydroxy-3-methoxyphenyl)butan-2-one, and 4-(3,4-dihydroxyphenyl)butan-2-one. Hydroxyphenylbutan-2-ols for use in the invention include, for example and without limitation, 4-(4-hydroxyphenyl)butan-2-ol, 4-(4-hydroxy-3-methoxyphenyl)butan-2-ol, and 4-(3,4-dihydroxyphenyl)butan-2-ol.

Compounds of formulas (1) and (2) may be obtained by extraction from natural materials. For instance, raspberry ketone glucoside is extracted from appropriate fruits (e.g., raspberry fruit) by solvent and is used as a concentrated form (Phytochemistry, 29, 12, 3853-3858, 1990).

Compounds of formulas (1) and (2) may also be synthesized using methods well known in the art, such as described in Japanese laid open applications P2000-169325 and P2001-22623, and PCT application PCT/JP02/05090, published as WO 02/096399. Additional methods for synthesis of compounds of formulas (1) and (2) may be found in European patent application EP 0 516 082.

Preferred compounds of the invention are raspberry ketone, zingerone and BK-180 which have high stability and are easily and conveniently obtained.

Compounds of formula (1) and compounds of formula (2) may be included in the compositions of the present invention singly or combined. The preferred total amounts of the compounds of formulas (1) and/or (2) are in the range of about 0.001 to about 30.0 weight % of the total composition, which is an optimal range for sufficient activity for body weight loss and modulation of unpleasant taste and aroma characteristics. More preferably, the total amount of the compounds of formulas (1) and/or (2) are in the range of about 0.03 to about 20.0 weight % of the total composition, and, most preferably, the total amount of the compounds of formulas (1) and/or (2) are in the range of about 0.1 to about 15.0 weight % of the total composition. A particular preferred total amount of the compounds of formulas (1) and/or (2) is in the range of about 1.0 to about 10 weight % of the total composition.

As used herein, the term “effective amount” refers to an amount of a compound or an amount of a composition sufficient to have a measurable effect on a defined state or condition. In certain preferred embodiments, an “effective amount” of compounds of formula (1) and/or (2) is administered to a mammal to cause weight loss, fat loss, slimming, or any combination of these effects. In such embodiments, an “effective amount” may administered in single or multiple doses. In certain embodiments, compositions (e.g., extracts) are provided that mask one or more unpleasant characteristics of a compound of formula (1) and/or (2). Accordingly, an “effective taste-masking amount” of a composition is an amount sufficient to reduce an unpleasant characteristic of a compound of formula (1) or (2). In certain preferred embodiments, compositions are provided in an “effective taste-masking amount” to mask a bitter taste and/or unpleasant odor of a compound of formula (1) and/or (2). An “effective taste-masking amount” of a composition can be determined by a panel of trained flavorists, as set forth below.

Compounds of formula (1) and (2) have been proposed as skin care materials having an inhibitory activity of melanin formation (JP 2000-239143) and were found to have activity for enhancement of fat decomposition and inhibition of obesity through topical treatment (JP 2000-169325). Compounds of formulas (1) and (2) are presumed to promote weight loss via fat decomposition. Fat decomposition is enhanced by the increased contact and activity of hormone sensitive lipase on intracellular fat. Intracellular fat is covered by a layer of phospholipid, predominantly phosphatidyl choline, that inhibits contact by the lipase. Hormones that promote lipocatabolism, e.g., norepinephrine, enhance fat decomposition by countering the lipase-inhibiting action of phosphatidyl choline, thereby increasing affinity of the lipase for intracellular fat. (Okuda, et al., J. Lipid Res., 35:36-44, 1994 and Okuda, et al., J. Lipid Res., 35:1267-1273, 1994). Compounds of formulas (1) and (2) are presumed to enhance the affinity of hormone sensitive lipase to intracellular fat in the same fashion as fat decomposition hormones.

Compounds containing ester groups, wherein R² is —COR³ and R³ is C₂-C₂₀ alkyl, are hydrolyzed upon acid treatment to the active hydroxyl form of the ingredient. Such derivatives may be administered orally and are consequently hydrolyzed in the acid stomach environment to the active form.

Extracts and other Taste Masking Ingredients

In one aspect, the invention is drawn to a composition comprising one or both of a compound of formula (1) and a compound of formula (2) and one or more extracts to mask unpleasant taste and/or aroma characteristics of these compounds. Extracts that mask one or more unpleasant characteristics of a compound of formula (1) or (2) can be obtained from, for example and without limitation, oolong tea, puerh tea, roasted adlay tea, eucommia leaves, jasmine flower, banzakuro (guava fruit), barley, mate tea (Paraguay tea), rooibos tea, tinpi (matured peal of Citrus unshiu), juemingzi, sanzashi (Crataegus), soybean germ, ginger, ginseng, kouki tea, and yucca. The extracts may be used in compositions of the invention individually, or in combination. The compositions comprising a compound of formula (1) and/or formula (2) and an extract may be added to or formulated as a food, beverage, dietary supplement, medical food, pet food, or cosmetic. The forgoing are non-limiting examples.

Whole plants or their leaves, fruits (preferably, defatted seeds), bark, root, branch, or fermented plants are either dried and powdered, dried without powdering, or powdered without drying, and then extracted with solvent at room temperature or with heating or extracted using instruments, such as a soxhlet extractor. Solvents used for extraction, include, but are not limited water, methanol, ethanol, propanol, 1,3-butylene glycol, benzene, ethyl ether, chloroform, ethyl acetate, butyl acetate, acetone, or any combination of any of the foregoing.

Extractions that are liquid may be used directly in products. Preferably, the extractions are used as powders after freeze drying or spray drying. Extractions may be further purified by liquid-liquid extractions or adsorption chromatography.

Preferably, the compositions of the invention comprise one or more extracts of oolong tea, puerh tea, roasted adlay tea, eucommia leaves, and jasmine flower. More preferably, the compositions of the invention comprise a combination of these extracts. A combination of extracts may comprise, for example and without limitation, 10 parts of oolong tea extract, 2-4 parts of puerh tea extract, 2-4 parts of roasted adlay tea extract, 1-3 parts of eucommia leaf extract, and 1-3 parts of jasmine flower extract. These extracts are commercially available, such as, for example, from KGPP-P1 (Life Food Research).

The extracts in the compositions of the invention are about 0.001-40.0 weight % of the total weight of the composition, which is an optimal range for achieving sufficient masking of undesirable taste and aroma characteristics of a compound of formula (1) or (2). Preferably, the total amount of the extract is about 0.01-20.0 weight % of the total weight of the composition.

Unpleasant taste and aroma characteristics of compounds of formula (1) and compounds of formula (2) may also be masked in compositions by including specific compounds, e.g., one or more compounds selected from the group consisting of malthol, ethyl maltol, furaneol, furaneol derivatives, vanillin, and ethyl vanillin. The compositions comprising a compound of formula (1) and/or formula (2) and any of the foregoing compounds, alone or in combination, may be added to or formulated as a food, beverage, dietary supplement, medical food, pet food, or cosmetic. The forgoing are non-limiting examples.

Clathrate Complexes

Unpleasant taste and aroma characteristics of compounds of formula (1) and (2) may also be masked by including the compounds in a clathrate. When present in clathrates, compounds of formula (1) and (2) also exhibit increased solubility, facilitating formulating compositions comprising increased amounts of compounds of formula (1) and (2) and more uniform distribution of the compound.

Specifically, the invention provides clathrates of any of hydroxyphenylbutan-2-ones of formula (1) or hydroxyphenylbutan-2-ols of formula (2) included in cyclodextrin. The invention further provides compositions that contain these clathrates in, for example and without limitation, a food, beverage, dietary supplement, medical food, pet food, cosmetic, bath salt, or the like.

Hence, in certain embodiments, the invention is directed to compositions comprising a clathrate comprising of a compound of formula (1) or formula (2). In a preferred embodiment, a compound of formula (1) or formula (2) forms part of a clathrate with a CD. CDs for use in the invention may be any α-CD, β-CD, γ-CD, as well as branched CDs, such as those with one or two oligosaccharide molecules of glucose, maltose, maltotriose, or the like α-1,6-bonded thereto, and chemically-modified branched CD such as hydroxypropyl-CD. Preferably, CD for use in the invention is β-CD or branched β-CD. One or more different types of such CDs may be used in the invention either singly or as combined.

CD compounds are known in the art (see, for example, Hileman et al., Electrophoresis 1998, 19(15):2677-2681). The compounds are generally defined as a cyclic ring of 1,4 linked glucose residues. CD compounds are typically classified based on the number of 1,4 linked glucose residues present in the ring, with rings of between 6 and 12 glucose residues being preferred. Rings of 6, 7, and 8 glucose residues are particularly preferred. Thus, CD compounds that comprise a ring of six 1,4 linked glucose residues (i.e., n=6) are referred to as α-CD compounds. CD compounds that comprise a ring of seven 1,4 linked glucose residues are referred to as β-CD compounds, and cyclodextrin compounds that comprise a ring of eight 1,4 linked glucose residues are referred to as γ-cyclodextrin compounds. The glucose residues may be substituted with chemical moieties to form cyclodextrin derivative compounds such as, without limitation, sulfated cyclodextrin or sulfonated cyclodextrins. CD compounds are commercially available (e.g., from Sigma-Aldrich, Milwaukee, Wis.) or may be synthesized using methods well known to one of ordinary skill in the art (see, e.g., Easton et al., Modified Cyclodextrins: Scaffolds and Templates for Supramolecular Chemistry, 304 pp., World Scientific, April 1999)

A clathrate of the invention may be produced by contacting any of hydroxyphenylbutan-2-ones or hydroxyphenylbutan-2-ols of formula (1) or formula (2) with CD in water to thereby form a clathrate in CD. The resulting clathrate may be further processed by drying and powdering, using methods well known in the art.

To form clathrates, typically CD is dissolved in water and a compound of formula (1) or formula (2) is added thereto and then vigorously stirred or shaken for a period of from a few seconds to a few hours, for example, in a stirrer or a homogenizer. Alternative means of forming clathrates include, without limitation, shaking in a closed vessel or ultrasonic treatment. During the process, crystals of a compound of formula (1) or formula (2) may be added to the CD solution directly in solid form. Alternatively, a compound of formula (1) or (2) may be added in solution, by first dissolving the compound in a suitable organic solvent and then adding the solution comprising the dissolved compound to the CD solution. Examples of suitable organic solvents for forming solutions comprising a compound of formula (1) or (2) are, without limitation, acetone, ethanol, methanol, isopropanol, tetrahydrofuran, and ethyl acetate. Especially preferred is acetone. The amount of the organic solvent to be used is that amount sufficient to dissolve the particular compound of formula (1) or formula (2) to be added to CD. The ratio of a compound of formula (1) or formula (2) to be added to CD generally ranges from about 0.1:1 to about 2:1 (mol/mol). Preferably, the ratio of a compound of formula (1) or formula (2) to be added to CD is in the range of about 0.5:1 to about 1:1 (mol/mol). The contact reaction temperature generally ranges from about 0 to about 100° C., preferably from about 5 to about 70° C. The clathrate comprising a compound of formula (1) and/or formula (2) thus obtained is typically dried and powdered.

The clathrates of the invention are not a mere admixture but are an inclusion bodies of a compound of formula (1) and/or formula (2) in CD. Formation of the inclusion body can be confirmed using methods well known in the art such as, for example and without limitation, differential calorimetry with a differential scanning calorimeter (DSC).

Clathrates comprising a compound of formula (1) or formula (2) do not have the unpleasant aroma peculiar to un-complexed forms of compounds of formula (1) and/or formula (2). Accordingly, formation of clathrates comprising compounds of formula (1) and/or formula (2) can be determined through organoleptic testing.

The CD-clathrates of the invention of any of a compound of formula (1) and/or formula (2) in CD may be used in various forms. For example, they may be used as a powder or may also be used in a solution or suspension.

Encapsulation Methods

The invention is also drawn to a composition comprising one or both of a compound of formula (1) and a compound of formula (2) wherein the composition is encapsulated with a coating effective to mask the taste of the composition. Such encapsulated compositions may be added to or formulated as, for example and without limitation, a food, beverage, dietary supplement, medical food, pet food, or cosmetic.

In preferred embodiments, compositions comprising a compound of formula (1) and/or (2) is encapsulated by one of spray drying, agglomeration, spray cooling, glass-like encapsulation, entrapping the material in a sugar melt or sugar alcohol melt, formation of a gelatin coating via formation of a coascervate, absorption, or entrapping in a liposome. In a further preferred embodiment, the coating comprises one or more extract selected from extracts of oolong tea, puerh tea, roasted adlay tea, eucommia leaves, jasmine flower, banzakuro (guava fruit), barley, mate tea (Paraguay tea), rooibos tea, tinpi (matured peal of Citrus unshiu), juemingzi, sanzashi (Crataegus), soybean germ, ginger, ginseng, kouki tea, and yucca. More preferred, the coating comprises one or more extract selected from extracts of oolong tea, puerh tea, roasted adlay tea, eucommia leaves, and jasmine flower. In another preferred embodiment, the coating comprises one or more of malthol, ethyl maltol, furaneol, furaneol derivatives, vanillin, and ethyl vanillin. Preferably, the coating further comprises a sugar alcohol. Preferably, the coating further comprises one or both of L-histidine and proline.

Compositions

It is desirable that the ratio of compounds of formulas (1) and/or (2) to extract is in the range of about 10:1 to about 1:20. More preferably, the ratio is in the range of about 1:1 to about 1:5. For compositions that are included in beverages and chewable food, the preferred ratio is from about 5:1 to about 1:1 to suppress the bitter and harsh taste of the compounds.

The compositions of the invention may include other ingredients. For instance, sugars may be added to enhance the masking effect. There is no limitation to the amount of the sugar added if the composition is used in food. Sugars that may be added include, but are not limited to, monosaccharides (for example, galactose and glucose), disaccharides (for example, sugar and trehalose), and sugar alcohols (for example, xylitol, lactitol, mannitol, sorbitol, maltitol, and erythrytol). In order to further sweeten the composition, one or more of these sugars may be used in combination. Appropriate sugars may be selected by sweetness, solubility, and texture. Preferably, sugar alcohols are used. In solid foods, the sugar content is preferably from about 15 to about 95 weight % and more preferably from about 50 to about 80 weight %, based on the weight of the composition. In liquids or gels, the sugar content is preferably from about 1 to about 20 weight % and more preferably from about 2 to about 10% weight %, based on the weight of the total composition. In this range, appropriate sweetness is obtained and balance of flavor is good.

It is further desirable to include an additional ingredient effective for weight loss. For example, materials to inhibit sugar and fat absorption may be added, which include, but are not limited to, gymunema extract, garcinia extract, banaba extract, salacia, albumin extracted form wheat, chitosan, “asia” polyphenol, rice germ extract, forskohlii powder, saponin, or any combination of any of the foregoing. Materials to enhance fat deposition may be added, which include, but are not limited to, conjugate linoleic acid, citrus, arurentium extract, or any combination of the foregoing. Materials to enhance fat burning may be added, which include, but are not limited to, soybean peptide, arginine, caffeine, capsaicin, ephedrine, piperine, carnitine, proline, or any combination of the forgoing. Materials to suppress appetite may be added, which include, but are not limited to, L-histidine, extracts from citrus such as grapefruit, or any of the foregoing. Materials to activate metabolism and enhance weight loss may be added, which include, but are not limited to, melilot extract, hucus extract, grape seed extract, ginkgo extract, adlay extract, or any of the foregoing.

Additional ingredients include, but are not limited to, gymunema extract, forskohlii powder, soybean peptide, arginine, adlay extract, L-histidine, and proline. Preferred additional ingredients are L-histidine and proline. The ratio of compounds of formulas (1) and/or (2) and total additional ingredients typically ranges from about 10:1 to about 1:5 and preferably, from about 5:1 to about 1:5.

It is further desirable to include vitamin C plant extracts, which are effective to reduce and inhibit melanin synthesis and to promote collagen synthesis, and hyaluronic acid and derivatives thereof for improvement of rough skin. Vitamin C is additionally desirable due to its role in the synthesis of adrenal cortical hormone and inhibition of mental stress.

Water soluble ascorbic acid and derivatives are the preferred forms of vitamin C used in the compositions of the invention. Water soluble ascorbic acid includes L-ascorbic acid and its sodium salt, potassium salt, magnesium salt, calcium salt, barium salt, ammonium salt, monoethanolamine salt, diethanolamine salt, triethanolamine salt, monoisopropanolamine salt, triisopropanolamine salt; L-ascorbic acid-2-phosphoric acid sodium salt, potassium salt, magnesium salt, calcium salt, barium salt, ammonium salt, monoethanolamine salt, diethanolamine salt, triethanolamine salt, monoisopropanolamine salt, triisopropanolamine salt; L-ascorbic acid-2-sulfuric acid sodium salt, potassium salt, magnesium salt, calcium salt, barium salt, ammonium salt, monoethanolamine salt, diethanolamine salt, triethanolamine salt, monoisopropanolamine salt, triisopropanolamine salt; and ascorbic acid sugar derivative such as L-ascorbic acid-2-o-glucoside. Water soluble ascorbic acid derivatives, L-ascorbic acid phosphoric acid sodium ester, L-ascorbic acid phosphoric acid magnesium ester, L-ascorbic acid-2-o-glucoside are also preferred. To increase the stability of vitamin C, it is preferred to use it with an organic acid, such as citric acid, malic acid, fumaric acid, malonic acid, succinic acid, tartaric acid, lactic acid, or any combination of any of the foregoing.

Additional ingredients that may be added to compositions of the present invention include plant extracts that reduce and inhibit melanin synthesis and promote collagen synthesis. Preferred extracts include extracts from licorice, camellia, peach, ziou, otogirisou (Hypericum erectum), loquat, or any combination of any of the foregoing. These extracts may be obtained from leaves, fruits, seeds (defatted), roots, or rhizome, through extraction by water, 1,3-butylene glycol, or mixture of these solvents. Hydrophobic extracts may be extracted by ethanol, concentrated, and then further extracted by squalane.

Further ingredients that may be added to the compositions of the invention include other vitamins and minerals. Vitamins include, but are not limited to vitamin B1, vitamin B2, niacin, pantothenic acid, vitamin B6, vitamin B12, folic acid, biotin, and inositol. Minerals include, but are not limited to, zinc, iron, calcium, magnesium, chromium, selenium, potassium, and sodium. Minerals that chelate with either an amino acid, such as aspartic acid, or an organic acid, such as gluconic acid or picric acid are preferred to enhance absorption.

The compositions may additionally contain royal jelly, salmon milt extract (DNA Na), yeast extract (RNA), or any combination of any of the foregoing, in an amount of from about 0.1 to about 10 weight % and more preferably, from about 1 to about 5 weight %, based upon the total weight of the composition.

Additional ingredients include organic acids such as, for example and without limitation, citric acid, malic acid, fumaric acid, malonic acid, succinic acid, tartaric acid, and lactic acid; dyes; synthetic or natural flavors such as, for example and without limitation, vanillin, linalool, raspberry flavor, apple flavor, and coffee flavor; drying agents; food fibers; electrolytes; antioxidants; preservatives; moisturizers; and any combination of any of the foregoing.

Food, Beverage, Dietary Supplement, Medical Food, Pet Food, or Cosmetic

Foods, beverages, dietary supplements, medical foods, and pet foods may be in the form of a tablet, granule, capsule, chewing gum, jelly, chocolate, candy, beverage, soup, ice cream, pasta, or baked good. The food is not particularly limited and may be a gel, powder, liquid, granule, cream, paste, or solid food. Cosmetics may be in the form of, for example, creams, milky lotions, lotions, essences, packs, cataplasms, foundations, face powders, lipsticks, and bath agents. Cosmetics include, but are not limited to, creams, lotions, pastes, gels and cakes that are suitable for application to the skin.

In a preferred embodiment, compounds of formulas (1) and/or (2) are formulated in a gel pack composition. The gel pack composition is applied to a release sheet to form a slimming sheet pack. The slimming sheet pack has superior properties, both as a cosmetic product and in terms of the ability to effect weight loss a fat loss compared to other forms of applying compounds of formulas (1) and/or (2). Hence, subjects that apply compounds of formulas (1) or (2) in the form of a gel pack experience greater fat loss and slimming, compared to subjects that apply compounds of formulas (1) or (2) in the form of paste.

Test Methods for Evaluation of Taste and Bitterness

Taste and bitterness can be evaluated using a panel of trained panelists (“flavorists”). A panel of potential flavorists is screened for their abilities to taste bitter substances. The panel is then trained to scale various bitter substances. The scaling requires that substances such as caffeine are used at various levels in a water solution. The panel is required to indicate the relative levels of bitterness in each solution. The panelists who can best scale the bitter substances are selected as flavorists to assess the bitterness of compounds and the masking capabilities of compounds and compositions.

When testing for bitterness in a food or beverage product, the following is standard procedure:

a. The sample of product with known bitterness is evaluated against a sample with a masking compound.

b. The expert panel evaluates the two samples for their degree of bitterness.

c. Typically the panel indicates if the bitterness is totally removed or significantly attenuated.

d. The panel may also be asked to indicate the quality of the flavor.

Methods

In certain embodiments, the invention is drawn to a method of reducing or controlling weight, by administering a clathrate including a compound of formula (1) and/or (2), or a composition comprising a compound of formula (1) and/or (2) and a component that masks unpleasant taste and aroma characteristics of these compounds. In preferred embodiments, the clathrate or composition is added to or formulated as a food, beverage, dietary supplement, medical food, pet food cosmetic or bath salt that is administered to a mamma, preferably a human, dog, or cat, to reduce or control weight or fat.

Effective amounts of the compounds of formulas (1) and/or (2) are tailored to the user's condition. There is no particular upper limit for consumption amount and frequency per day, however preferably, a range of about 50 to about 300 mg/day/ 70 kg body weight (adult) is administered. There is also no limitation on time of administration, however, preferably, the administration occurs before a meal.

The following Examples are provided for a further illustration of the invention, but are not intended to be limiting thereof. Unless otherwise indicated, all parts and percentages are by weight of the final composition.

### EXAMPLE 1

Granule samples, Sample 1, Sample 2, and Sample 3, were made based on the formulas listed in Table 1 by conventional procedure. The ingredients shown in Table 1 were mixed with alcohol. The resulting mixture was granulated through an extrusion granulator having a 0.8 mm diameter extrusion orifice. The resulting granules were dried in a batch-type flow drier with charge air temperature of 70° C. and exhaust gas temperature of 50° C. for 10 min, until the water content of the dried granules was reduced to 7% or less.

TABLE 1
Formulations of Sample 1, Sample 2,
and Sample 2 by weight %.

| Materials | Sample 1 | Sample 2 | Sample 3 |
|---|---|---|---|
| Raspberry ketone | 6 | 6 | 6 |
| Oolong tea (dry) | 1.2 | 0.3 | 39 |
| Puerh tea (dry) | 0.3 | 0.1 | 9.75 |
| Roasted adlay tea (dry) | 0.3 | 0.1 | 9.75 |
| Eucommia leaves (dry) | 0.1 | 0.025 | 3.25 |
| Jasmine flower (dry) | 0.1 | 0.025 | 3.25 |
| Maltitol | 76.4 | 77.85 | 13.4 |
| Suclarose | 0.1 | 0.1 | 0.1 |
| Raspberry flavorant | 5 | 5 | 5 |
| Excipient (“seorasu”) | 10 | 10 | 10.0 |
| Flavor | 0.5 | 0.5 | 0.5 |
| Total | 100 | 100 | 100 |

Sample 1 and comparative samples, Sample 2 and Sample 3, were tasted by 18 volunteers. Bitter and harsh flavor was evaluated by trained volunteers, and the results were summarized in Table 2.

TABLE 2
Results of taste test.

| | Sample 1 | Sample 2 | Sample 3 |
|---|---|---|---|
| Bitter, harsh | ◯ | X | Δ |
| taste reported | | | |

Flavor improvement key:

9 or more volunteers answered they tasted a bitter, harsh taste—X

5-8 volunteers answered they tasted a bitter, harsh taste—Δ

1-4 volunteers answered they tasted a bitter, harsh taste—O

### EXAMPLE 2

A chewable type tablet, Sample 4, was formulated based on the ingredients of Table 3. The ingredients shown in Table 3 were mixed uniformly. Using a tabletting machine, the resulting mixture powder was directly continuously tabletted into tablets having a weight of 0.5 g. The tabletting speed was 12 tablets/min and pressure was 2 t/m². A total of approximately 180,000 (approximately 9 kg) tablets were produced.

TABLE 3
Formulation of Sample 4 by weight %.

| Materials | Sample 4 |
|---|---|
| Raspberry ketone | 5.6 |
| Oolong tea (dry) | 0.72 |
| Puerh tea (dry) | 0.2 |
| Roasted adlay tea (dry) | 0.2 |
| Eucommia leaves (dry) | 0.16 |
| Jasmine flower (dry) | 0.16 |
| “Banzakuro” (dry) | 0.11 |
| Barleyl (dry) | 0.08 |
| Mate tea (dry) | 0.08 |
| Rooibos tea (dry) | 0.08 |
| “Tinpi” (dry) | 0.06 |
| Juemingzi (dry) | 0.05 |
| “Sanzashi” (dry) | 0.03 |
| Soybean germ (dry) | 0.03 |
| Ginger (dry) | 0.02 |
| Ginseng extract (dry) | 0.01 |
| “Kouki” tea extract (dry) | 0.01 |
| Yucca extract (dry) | 0.01 |
| gymunema extract (dry) | 1 |
| Yucca saponin (dry) | 1 |
| Adlay extract powder (dry) | 1 |
| L-histidine | 0.5 |
| Forskohlii powder (dry) | 0.1 |
| Vitamin C | 34.5 |
| Hyaluronic acid | 0.1 |
| Vitamin B6 | 0.1 |
| Vitamin P | 0.5 |
| Inositol | 11.2 |
| maltitol | 20.5 |
| Sucrose | 0.5 |
| Salmon milt extract (DNA Na) | 0.1 |
| Yeast extract (RNA) | 0.1 |
| excipient (“seorasu”) | Rest |
| Raspberry flavor | 5.7 |
| total | 100 |

The flavor of the chewable tablet was tested according to the procedures given in Example 1. The number of testers indicating bitterness and acridity was 2 out of 18.

### EXAMPLE 3

The ingredients in Table 4 were put into a chewing gum mixer and mixed for 15 min, until homogeneous. The mixture was rolled and cut into chewing gum weighing 3 g (20×14×11 mm).

TABLE 4
Formulation of Sample 5 by weight %.

| Materials | Sample 5 |
|---|---|
| Gum base | 20 |
| Maltitol | 62 |
| Raspberry ketone | 5 |
| Jasmine flower (dry) | 0.9 |
| Roasted adlay (dry) | 1.1 |
| gymunema extract (dry) | 0.5 |
| Adlay extract powder (dry) | 0.5 |
| Vitamin C | 5 |
| Reduced maltose starch syrup | 4 |
| Raspberry flavor | 1 |
| Total | 100 |

The flavor of the chewing gum (3 g/stick) was tested according to the procedures given in Example 1. Test results were good, the number of testers indicating bitterness and acridity were 0 out of 18.

### EXAMPLE 3B

Raspberry flavor can also be added sugarless gums according to the following compositions:

| | | | |
|---|---|---|---|
| | | Fruit | Fruit |
| | | Acid | Acid |
| Stick | Bubble | Stick | Bubble |
| Gum | Gum | Gum | Gum |

| | | | | |
|---|---|---|---|---|
| Dreyfus Base | 25.0 | | 25.0 | |
| Dreyfus bubble base | | 26.0 | | 26.0 |
| 70% Sorbitol solution | 15.0 | 17.0 | 15.0 | 17.0 |
| 100-Mesh Powdered | | | | |
| Sorbitol | 47.5 | 48.3 | 47.0 | 47.3 |
| Glycerine | 5.0 | 2.0 | 5.0 | 2.0 |
| Fruit acid | | | 1.0 | 1.0 |
| Flavor | 1.3 | 0.5 | 0.8 | 0.5 |
| Raspberry ketone | 6.0 | 6.0 | 6.0 | 6.0 |
| Aspartame | 0.2 | 0.2 | 0.2 | 0.2 |
| | 100.0% | 100.0% | 100.0% | 100.0% |

The sugarless gum formulations set forth above are put into a chewing gum mixer and mixed for 15 min, until homogeneous. The mixture is rolled and cut into chewing gum weighing 3 g (20×14×11 mm). The flavor of the sugarless gum is tested according to the procedures given in Example 1.

### EXAMPLE 4

Gel beverage (200 ml/pack), Sample 6, was formulated based on the ingredients listed in Table 5 using a conventional method. The ingredients given in Table 5 were mixed and homogenized to prepare a gel dispersion. The dispersion was cooled to about 10° C. and was filled into 200 ml pouches which were then sealed. The pouches were then thermally sterilized by showering with hot water (67° C., 35 min), and then were cooled to 35° C. or lower. The process yields a jelly drink.

TABLE 5
Formulation of Sample 6 by weight %.

| Materials | Sample 6 |
|---|---|
| Raspberry ketone | 0.3 |
| Puerh tea (dry) | 0.04 |
| Eucommia tea (dry) | 0.02 |
| Garcinia extract (dry) | 1 |
| Maltodextrin | 1.5 |
| Reduced maltose starch syrup | 1.25 |
| Galactose | 1 |
| Carrageenan | 0.9 |
| Concentrated fruit | 0.5 |
| Citric acid | 0.4 |
| Suclarose | 0.003 |
| Flavor | 0.1 |
| Water | 92.987 |
| Total | 100 |

The flavor of the jelly drink (200 ml) was tested according to the procedures given in Example 1. Test results were good, the number of testers indicating bitterness and acridity were 1 out of 18.

### EXAMPLE 5

Beverage (100 ml), Sample 7, was formulated based on the ingredients listed in Table 6. The ingredients listed in Table 6 were added into 50 mL of water. Once the ingredients dissolved, water was added to 100 mL total beverage.

TABLE 6
Formulation of Sample 7 by weight %.

| Materials | Sample 7 |
|---|---|
| Raspberry ketone | 0.2 |
| Oolong tea (dry) | 0.03 |
| Jasmine flower (dry) | 0.005 |
| Mate tea (dry) | 0.005 |
| L-histidine | 1 |
| Adlay extract powder (dry) | 0.1 |
| Citric acid | 1 |
| Sodium citrate | 0.5 |
| Vitamin B2 | 0.01 |
| Vitamin B6 | 0.01 |
| Pantothenic acid | 0.01 |
| Nicotinic acid | 0.005 |
| Maltitol | 5 |
| Saccharin | 0.02 |
| Sucrose fatty acid ester | 10 |
| Flavor | Small amount |
| Water | Rest |
| Total | 100 |

The flavor of the beverage (100 ml) was tested according to the procedures given in Example 1. Test results were good, the number of testers indicating bitterness and acridity were 0 out of 18.

### EXAMPLE 6

Hard candy, Sample 8, was formulated based on the ingredients listed in Table 7 using a conventional method. Reducing palatinose and trehalose were heated in a still with sufficient water until completely dissolved. The mixture was then boiled in a cooker until water content was reduced to 2.5%. The reduced mixture was then mixed with the ingredients listed in Table 7. The resulting mixture was then cooled, sized in a batch former, and stamped into hard candies.

TABLE 7

| Material | Sample 8 |
|---|---|
| Raspberry ketone | 6 |
| Roasted adlay | 3.5 |
| Jasmine flower | 2.5 |
| Reduced paratinose | 40 |
| Trehalose | 43 |
| Lemon juice | 3 |
| Citric acid | 1.5 |
| Sweetener (stevia) | 0.2 |
| Flavor | 0.3 |
| Total | 100 |

The hard candy is tested according to the procedures given in Example 1. Test results were good, with 0 out of 18 testers indicating bitterness or acridity.

In the following examples, Proton Nuclear Magnetic Resonance Spectrum (¹H-NMR) was determined using an AM-400 apparatus(400 MHz; Bruker) with tetramethylsilane for external standard. Differential Scanning Calorimeter (DSC) was performed using a DSC-220 apparatus (Seiko Electronic Industry).

### EXAMPLE 7

Preparation of 4-(4-hydroxyphenyl)butan-2-one (hereinafter referred to as raspberry ketone)/β-CD clathrate

In a nitrogen atmosphere, 69.1 g (60.9 mmol) of β-CD (Nihon Shokuhin Kako) and 800 ml of pure water were put into a 2-liter four-neck flask equipped with a Dimroth condenser and a thermometer, and dissolved by stirring at about 60° C. After it was confirmed that the system gave a clear solution, 10 g (60.9 mmol) of raspberry ketone (Takasago International) dissolved in acetone (30 ml) was dropwise added thereto, and stirred for 30 minutes at that temperature. Heating was stopped, and the solution was gradually cooled still with stirring, then cooled with ice, and further stirred at a temperature not higher than 5° C. for 1 hour. Thus deposited, the resulting clathrate was taken out through filtration under reduced pressure, washed with 200 ml of water cooled with ice, and then dried. The process gave 63.2 g of a white powder, raspberry ketone/β-CD clathrate.

The proton nuclear magnetic resonance spectrum (¹H-NMR) of the thus-obtained clathrate (in a solvent of dimethylsulfoxide deuteride, (CD₃)₂SO) was measured. This confirmed that the molar ratio of β-CD/raspberry ketone in the clathrate was about 1:0.91 and that 1000 mg of the clathrate contained 108 mg of raspberry ketone.

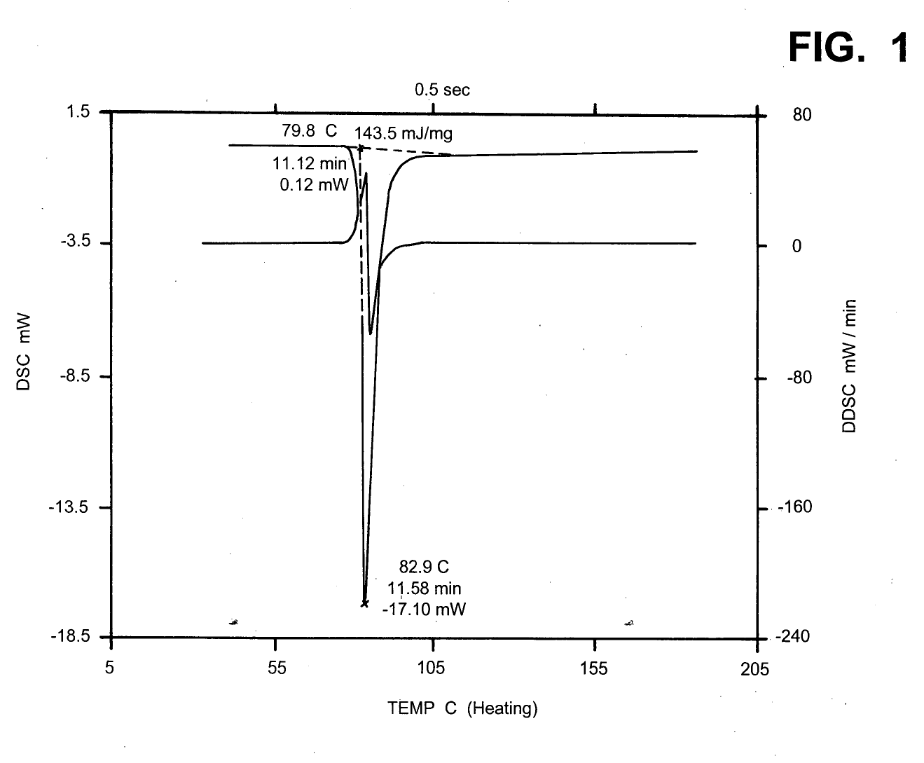

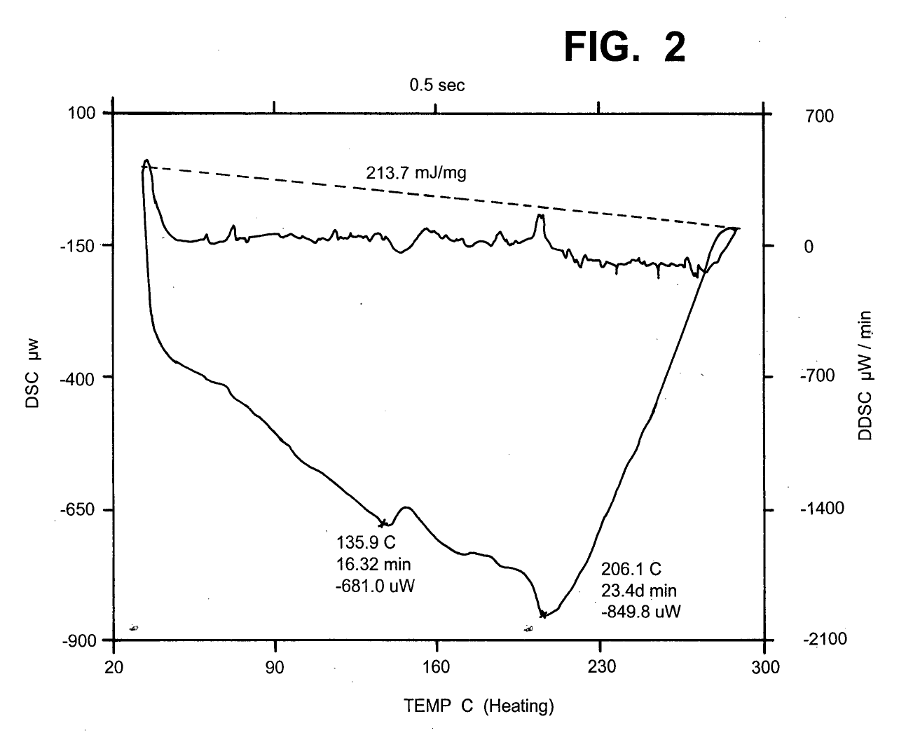

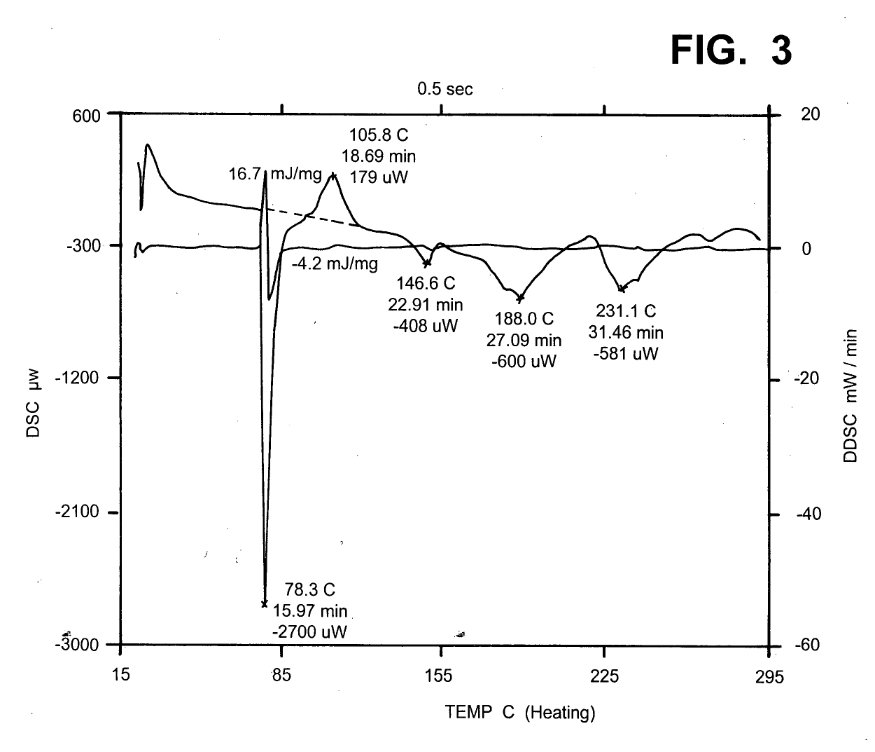

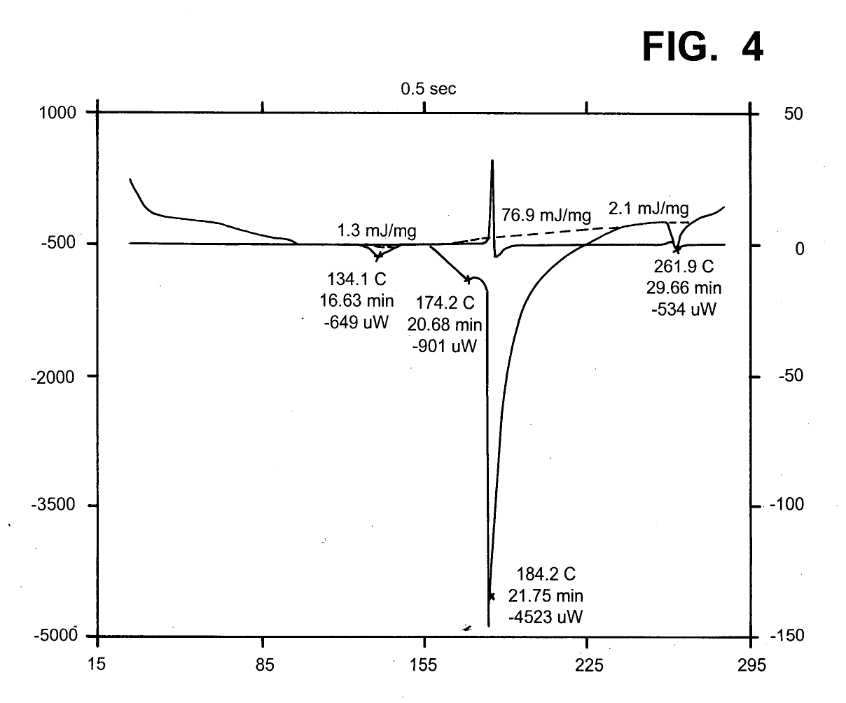

To confirm that the compound obtained herein was not a mere mixture but was a true clathrate body, a mixture of β-CD and raspberry ketone was prepared in a ratio of 9:1 by weight. Four samples, raspberry ketone alone, β-CD alone, the mixture of the two, and the clathrate of the two were compared through differential scanning calorimetry. FIGS. 1 to 4 are DSC patterns of the samples heated at 10° C./min. FIG. 1 is a DSC pattern of raspberry ketone alone; FIG. 2 is a DSC pattern of β-CD alone; FIG. 3 is a DSC pattern of the mixture of β-CD and raspberry ketone in a ratio of 9:1 by weight; and FIG. 4 is a DSC pattern of the clathrate obtained in this Example 7.

FIGS. 1-4 were compared with each other. An endothermic peak (at 78 to 80° C.) is seen in FIG. 3 but not in FIG. 4. This confirms that the compound prepared in this Example was a clathrate of raspberry ketone in β-CD.

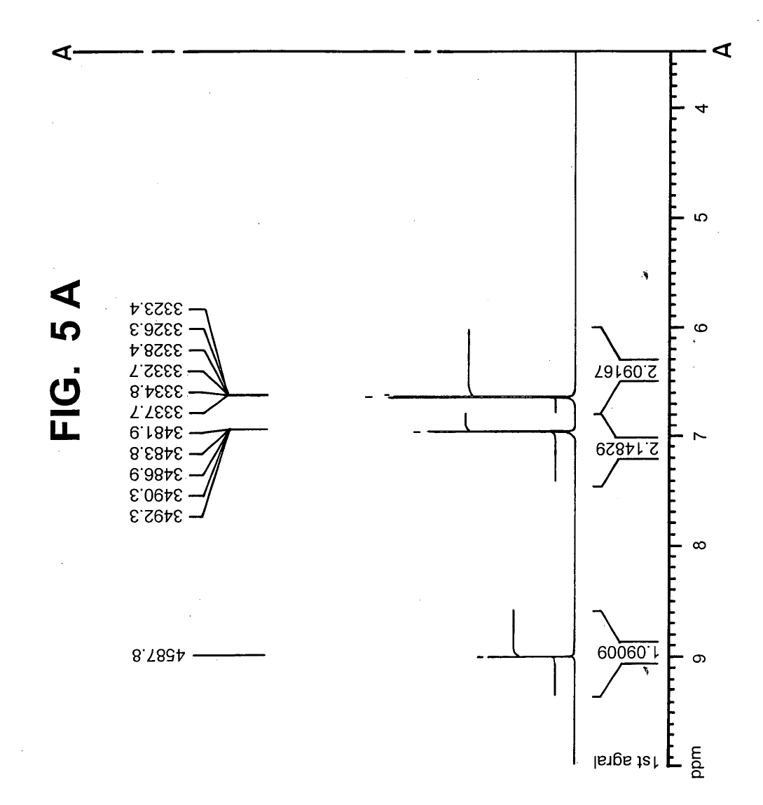

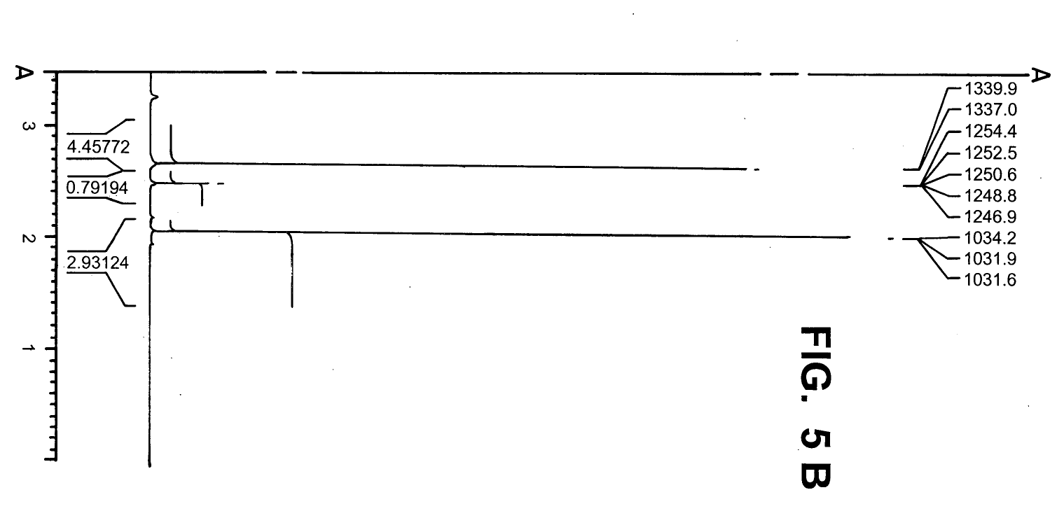

FIGS. 5 and 6 each are ¹H-NMR spectra [in (CD₃)₂SO] of raspberry ketone alone and that of the compound prepared in this Example (that is, raspberry ketone/β-CD clathrate), respectively, relative to an external standard, tetrainethylsilane (TMS). The clathrate was processed with chloroform to extract the included raspberry ketone, and the extracted raspberry ketone was analyzed using ¹H-NMR. Raspberry ketone extracted from clathrate had the same ¹H-NMR pattern as raspberry ketone that had not been included and exracted from clathrates. These results indicated that incorporation of raspberry ketone into clathrate complexes did not lead to its degradation.

### EXAMPLE 8

Preparation of 4-(3,4-dihydroxyphenyl)butan-2-one/β-CD clathrate

In a nitrogen atmosphere, 62.4 g (55 mmol) of β-CD (Nihon Shokuhin Kako) and 720 ml of pure water were put into a 2-liter four-neck flask equipped with a Dimroth condenser and a thermometer, and dissolved by stirring at about 60° C. After it was confirmed that the system gave a clear solution, 9.0 g (50 mmol) of 4-(3,4-dihydroxyphenyl)butan-2-one dissolved in acetone (27 ml) was dropwise added thereto, and stirred for 30 minutes at that temperature. Heating was stopped, and this was gradually cooled still with stirring, then cooled with ice, and further stirred at a temperature not higher than 1 to 3° C. for 1 hour. Thus deposited at that temperature, the resulting clathrate was taken out through filtration under reduced pressure, washed with 200 ml of water cooled with ice, and then dried. The process gave 57.5 g of a white powder. The proton nuclear magnetic resonance spectrum (¹H-NMR) of the thus-obtained clathrate (in a solvent of dimethylsulfoxide deuteride, (CD₃)₂SO) was measured. This confirmed that the molar ratio of β-CD/4-(3,4-dihydroxyphenyl)butan-2-one in the clathrate is about 1/0.88 and 1000 mg of the clathrate contains 121 mg of 4-(3,4-dihydroxyphenyl)butan-2-one.

### EXAMPLE 9

Sample 9 and Comparative Samples 9a and 9b

Using the clathrate of raspberry ketone/β-CD that had been prepared in Example 7, gum (Sample 9) was produced as in Table 8 that indicates its formulation (% by weight). For comparison, non-clathrated raspberry ketone alone (Comparative sample 9a), and a mixture of raspberry ketone and ,β-CD (Comparative sample 9b) was used in place of the clathrate in producing other samples of gum as in Table 8. Ten expert panelists tried all these gum samples to organoleptically evaluate them. The test results are given in Table 8.

TABLE 8

| | Sample 9 | Comp. Sample 9a | Comp. Sample 9b |
|---|---|---|---|
| Gum Base | 20 | 20 | 20 |
| Lactitol | 60 | 60 | 60 |
| Maltitol | 12 | 15.3 | 12 |
| Reducing Glutinous | 3 | 3 | 3 |
| Starch Syrup | | | |
| Fragrance | 1.2 | 1.2 | 1.2 |
| Grape Skin Dye | 0.1 | 0.1 | 0.1 |
| Raspberry Ketone | 3.7 | — | — |
| Clathrate | | | |
| Raspberry Ketone | — | 0.4 | 0.4 |
| β-cyclodextrin | — | — | 3.3 |
| Evaluation (taste) | good | strong smell of | strong smell of |
| | | raspberry ketone | raspberry ketone |

### EXAMPLE 10

Sample 10 and Comparative Samples 10a and 10b

Using the clathrate of raspberry ketone/β-CD that had been prepared in Example 7, tablet candies (Sample 10) were produced from the formulations in Table 9, using a continuous tabletting machine. For comparison, non-clathrated raspberry ketone alone (Comparison sample 10a), and a mixture of raspberry ketone and β-CD (Comparison sample 10b) were used in place of the clathrate in producing other tablet candies as in Table 9. In the same manner as in Example 9, the tablet candies were organoleptically evaluated by panelists. In the process of producing the tablet candies, the starting material was checked for its flowability in the hopper of the tabletting machine used, and, in addition, the tablet candies were checked for separation of raspberry ketone therein. The test results are given in Table 9.

TABLE 9

| | Sample 10 | Comp. Sample 10a | Comp. Sample 10b |
|---|---|---|---|
| Sorbitol | 45 | 90 | 45 |
| Sucrose Ester | 3 | 3 | 3 |
| Fragrance | 1.3 | 1.3 | 1.3 |
| Sour Seasoning | 0.7 | 0.7 | 0.7 |
| Raspberry Ketone | 50 | — | — |
| Clathrate | | | |
| Raspberry Ketone | — | 5 | 5 |
| β-cyclodextrin | — | — | 45 |
| Evaluation | | | |
| (taste) | good | strong smell of | strong smell of |
| | | raspberry ketone | raspberry ketone |
| (workability, | ◯ | Δ | Δ |
| flowability) | good | bridges formed in | bridges formed in |
| | | hopper | hopper |
| (workability, | no | separation found | separation found |
| separation of | separation | | |
| raspberry ketone | | | |
| in tablets) | | | |

### EXAMPLE 11

Sample 11 and Comparative Samples 11a and 11b

Using the clathrate of raspberry ketone/β-CD that had been prepared in Example 7, powder for drink (Sample 11) was produced as in Table 10 that indicates its formulation (% by weight). For comparison, non-clathrated raspberry ketone alone (Comparative sample 11a), and a mixture of raspberry ketone and β-CD (Comparative sample 11b) were used in place of the clathrate in producing other samples of powder for drink as in Table 10. 20 g of each powder was dissolved in 150 ml of water, and panelists tried the resulting drinks to organoleptically evaluate them in the same manner as in Example 9. While dissolved in water, each powder was visually checked for its solubility. The test results are given in Table 10.

TABLE 10

| | | | Comp. | Comp. |
|---|---|---|---|---|
| | | Sample 11 | Sample 11a | Sample 11b |
| Sugar | | 75 | 79.5 | 75 |
| Sour Seasoning | | 7 | 7 | 7 |
| Fragrance | | 4 | 4 | 4 |
| Branched Amino Acid | | 8 | 8 | 8 |
| Vitamin C | | 1 | 1 | 1 |
| Raspberry Ketone | | 5 | — | — |
| Clathrate | | | | |
| Raspberry Ketone | | — | 0.5 | 0.5 |
| β-cyclodextrin | | — | — | 4.5 |
| Evaluation | (taste) | good | strong smell of | strong smell of |
| | | | raspberry ketone | raspberry ketone |
| | (solubility | OO | x | x |
| | in water) | good | raspberry ketone | raspberry ketone |
| | | | remained | remained |
| | | | undissolved | undissolved |

### EXAMPLE 12

Sample 12 and Comparative Samples 12a and 12b

Using the clathrate of raspberry ketone/β-CD that had been prepared in Example 7, drink (Sample 12) was produced as in Table 11 that indicates its formulation (% by weight). For comparison, non-clathrated raspberry ketone alone (Comparative sample 12a), and a mixture of raspberry ketone and β-CD (Comparative sample 12b)were used in place of the clathrate in producing other samples of drink as in Table 11. Panelists tried the drink samples to organoleptically evaluate them in the same manner as in Example 9. In addition, the drink samples were visually checked as to whether or not raspberry ketone was completely dissolved therein. The test results are given in Table 11.

TABLE 11

| | | | Comp. | |
|---|---|---|---|---|
| | | Sample 12 | Sample 12a | Comp. Sample 12b |
| Erythritol | | 90 | 90.9 | 90 |
| Raspberry Juice | | 7 | 7 | 7 |
| Vitamin C | | 0.3 | 0.3 | 0.3 |
| Fragrance | | 0.9 | 0.9 | 0.9 |
| Sour Seasoning | | 0.8 | 0.8 | 0.8 |
| Raspberry Ketone | | 1 | — | — |
| Clathrate | | | | |
| Raspberry Ketone | | — | 0.1 | 0.1 |
| β-cyclodextrin | | — | — | 0.9 |
| Evaluation | (taste) | good | strong smell of | strong smell of |
| | | | raspberry ketone | raspberry ketone |
| | (appear- | OO | x | x |
| | ance) | clear | raspberry ketone | raspberry ketone |
| | | | remained | remained |
| | | | undissolved | undissolved |

### EXAMPLE 13

Sample 13 and Comparative Samples 13a and 13b

Using the clathrate of raspberry ketone/β-CD that had been prepared in Example 7, dog food (Sample 13) was produced as in Table 12 that indicates its formulation (% by weight). For comparison, non-clathrated raspberry ketone alone (Comparative sample 13a), and a mixture of raspberry ketone and β-CD (Comparative sample 13b) were used in place of the clathrate in producing other samples of dog food as in Table 12. Panelists tried the dog food samples to organoleptically evaluate them in the same manner as in Example 9. The test results are given in Table 12.

TABLE 12
Example of Dog Food

| | | Comp. | Comp. |
|---|---|---|---|
| | Sample 13 | Sample 13a | Sample 13b |
| Cereals (wheat, corn) | 50.0 | 50.0 | 50.0 |
| Meat (meat meal) | 45.0 | 45.0 | 45.0 |
| Premix (vitamins, | 3.0 | 3.0 | 3.9 |
| minerals) | | | |
| Fragrance | 1.0 | 1.0 | 1.0 |
| Raspberry Ketone | 1.0 | — | — |
| Clathrate | | | |
| Raspberry Ketone | — | 0.1 | 0.1 |
| β-cyclodextrin | — | 0.9 | — |
| Evaluation (taste) | good | strong smell of | strong smell of |
| | | raspberry ketone | raspberry ketone |

### EXAMPLE 14

Sample 14 and Comparative Samples 14a and 14b

Using the clathrate of raspberry ketone/β-CD that had been prepared in Example 7, cat food (Sample 14) was produced as in Table 13 that indicates its formulation (% by weight). For comparison, non-clathrated raspberry ketone alone (Comparison sample 14a), and a mixture of raspberry ketone and β-CD (Comparison sample 14b) were used in place of the clathrate in producing other samples of cat food as in Table 13. Panelists tried the cat food samples to organoleptically evaluate them in the same manner as in Example 9. The test results are given in Table 13.

TABLE 13
Example of Cat Food

| | Sample 14 | Sample 14a | Sample 14b |
|---|---|---|---|
| Cereals (wheat, corn) | 50.0 | 50.0 | 50.0 |
| Meat (fish meal, meat | 45.0 | 45.0 | 45.0 |
| meal) | | | |
| Premix (vitamins, | 3.0 | 3.0 | 3.9 |
| minerals) | | | |
| Fragrance | 1.0 | 1.0 | 1.0 |
| Raspberry Ketone | 1.0 | — | — |
| Clathrate | | | |
| Raspberry Ketone | — | 0.1 | 0.1 |
| β-cyclodextrin | — | 0.9 | — |
| Evaluation (taste) | good | strong smell of | strong smell of |
| | | raspberry ketone | raspberry ketone |

### EXAMPLE 15

Nutrient Functional Drink, 50 ml Mini-drink

The constitutive components of the formulation of Table 14 above were prepared. The weight of each component is 100 times in Table 14. Except raspberry ketone clathrate and peach flavor, all the components were dissolved in about 3000 ml of pure water under heat. After heating the resulting composition was stopped, raspberry ketone clathrate was added to it and dissolved, and pure water was added thereto to be about 5000 ml in total. This was cooled, and peach flavor was added thereto at about 50° C., and pure water was further added thereto to be 5000 ml in total. This was filled into 50-ml glass vials.

TABLE 14

| Formulation (in 50 ml): | | |
|---|---|---|
| Raspberry ketone clathrate | 500 | mg |
| Gymnema extract | 150 | mg |
| Betaine | 500 | mg |
| Inositol | 500 | mg |
| Hardly-digestible dextrin | 2 | g |
| Soybean oligosaccharide | 500 | mg |
| Royal jelly | 100 | mg |
| Sodium riboflavin phosphate | 12.5 | mg |
| Pyridoxine hydrochloride | 50 | mg |
| Ascorbic acid | 30 | mg |
| Nicotinamide | 10 | mg |
| Erythritol | 2 | g |
| Sucralose (trichlorogalactose) | 10 | mg |
| Citric acid | 140 | mg |
| Malic acid | 100 | mg |
| Sodium benzoate | 20 | mg |
| Butyl parahydroxybenzoate | 1.5 | mg |
| Ethyl parahydroxybenzoate | 3.5 | mg |
| Peach flavor | 50 | μl |
| Pure water to make | 50 | ml in all |

### EXAMPLE 16

Nutrient Functional Food, Packaged Jelly

The constitutive components of the formulation of Table 15 above were prepared. The weight of each component is 100 times in Table 15. Except raspberry ketone clathrate and peach flavor, all the components were dissolved in about 1000 ml of pure water under heat. After heating the resulting composition was stopped, raspberry ketone clathrate and peach flavor were added to it and dissolved, and pure water was added thereto to be 1500 ml in total. While hot, this was packaged into aluminum-laminate sticks.

TABLE 15

| Formulation (in 15 g): | | |
|---|---|---|
| Raspberry ketone clathrate | 100 | mg |
| Hardly-digestible dextrin | 2 | g |
| Betaine | 500 | mg |
| Inositol | 500 | mg |
| Artichoke extract | 350 | mg |
| Sodium riboflavin phosphate | 3 | mg |
| Pyridoxine hydrochloride | 5 | mg |
| Ascorbic acid | 50 | mg |
| Nicotinamide | 10 | mg |
| Erythritol | 1.25 | g |
| Sucralose (trichlorogalactose) | 3 | mg |
| Sodium benzoate | 10 | mg |
| Gelling agent (premix of carrageenan, | 90 | μg |
| locust bean gum and agar) | | |
| Peach flavor | 7.5 | μl |
| Pure water to make | 15 | ml in all |

As is obvious from the Tables mentioned above, the clathrate of any of hydroxyphenylbutan-2-ones or hydroxyphenylbutan-2-ols in CD has a mild smell and is highly soluble in water, and its processability into preparations is good.

### EXAMPLE 17

Slimming Sheets

Gel pack compositions were prepared from the ingredients listed in Table 16. Gelatin was dissolved in 50 g of water, and polyvinyl alcohol in 50 g of water. These solution were combined and mixed with stirring while heating. Raspberry ketone and/or 4-(3′,4′-dihydroxy-phenyl)butan-2-ol was dissolved in a solution of 10 g of water and 10 g of ethanol. Sodium polyacrylate was then mixed with glycerin, and the resulting mixture and the other ingredients were mixed with stirring to obtain 1 kg of a gel pack composition. Gel pack compositions were then spread on one surface of a sheet substrate of nonwoven polyester fabric(100 mm×400 mm) having a weight of 100 g/m² to form a layer having a weight of 800 g/m². A release sheet was placed on the surface of the pack composition to complete the slimming sheet pack. The slimming sheet packs were evaluated for slimming activity and adhesiveness as described below.

Slimming sheet packs were evaluated by women subjects aged 30-49 (18 total). Each subject was given two different slimming sheets chosen from sheets formulated from samples 101-104. Slimming sheets were grouped in paired sets representing each of the six possible pairs of sheets possible for samples 101-104. Each of the six possible pairs for samples 101-104 was tested by three subjects. Each subject was given a set of 20 sheets (i.e., 10 each of the two different types of sheets in each paired set), to be applied over a ten day period. Subjects placed one type of sheet pack from the set they were given to the same site of the left thigh every night before bed, and removed it every morning upon awakening. The other type of sheet from the set was similarly applied and removed from a site on the right thigh. Subjects applied the sheet packs each night for 10 continuous days. At the end of the 10-day period, subjects measured the circumference of their left and right thighs, to evaluate the effect of slimming of the sheet pack they had tried. Subjects also evaluated the adhesiveness of the sheet pack to their skin, based on the criterion of whether the sheet pack remain attached to the skin. Nine panelists tried each slimming sheet formulated from samples 101-104.

Results are given in Table 16.

TABLE 16

| | Sample | Sample | | |
|---|---|---|---|---|
| | 101 | 102 | Sample 103 | Sample 104 |
| Raspberry ketone | 1.0 | 1.0 | 1.0 | 0.8 |
| 4-(3′,4′-Dihydroxy- | — | — | — | 0.2 |
| phenyl)butan-2-ol | | | | |
| Sodium polyacrylate | 7.8 | 10.0 | 7.8 | 8.5 |
| Polyvinyl alcohol | 4.2 | 2.0 | 4.2 | 3.5 |
| Carboxyvinyl polymer | 1.0 | 1.0 | 1.0 | 1.0 |
| Glycerin | 28.0 | 28.0 | 28.0 | 28.0 |
| Polyoxyethylene | 0.4 | — | 0.4 | 0.4 |
| sorbitan monooleate | | | | |
| (20 E.O.) | | | | |
| Titanium oxide | 0.15 | 0.15 | 0.15 | 0.15 |
| Anhydrous caffeine | 0.1 | 0.1 | — | 0.1 |
| Phaeophytes extract | 0.1 | 0.1 | — | 0.1 |
| (in terms of dry | | | | |
| residue) | | | | |
| Tea extract¹ | 0.1 | 0.1 | — | 0.1 |
| (in terms of dry | | | | |
| residue) | | | | |
| Ethanol | 2.0 | 2.0 | 2.0 | 2.0 |
| Tartaric acid | 0.05 | 0.05 | 0.05 | 0.05 |
| Methylparaben | 0.1 | 0.1 | 0.1 | 0.1 |
| Ethylparaben | 0.1 | 0.1 | 0.1 | 0.1 |
| Fragrance | 0.1 | 0.1 | 0.1 | 0.1 |
| Pure water | balance | balance | balance | balance |
| Masking effect² | A | A | C | B |
| Slimming effect³ | A | B | B | A |

¹KGPP-P1 (Life Food Research)
²Slimming effect:
A: 8 or 9 subjects reported a circumference reduction of at least 0.5 cm.
B: 5 to 7 subjects reported a circumference reduction of at least 0.5 cm.
³Masking effect:
A: 8 or 9 subjects reported masking of unpleasant odor of the sheet pack
B: 5 to 7 subjects reported masking of unpleasant odor of the sheet pack
C: 0 to 4 subjects reported masking of unpleasant odor of the sheet pack

The present invention is not to be limited in scope by the specific embodiments described herein. Various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference.

We claim:

1. A composition comprising:
(i) one or both of a compound of formula (1) or formula (2) 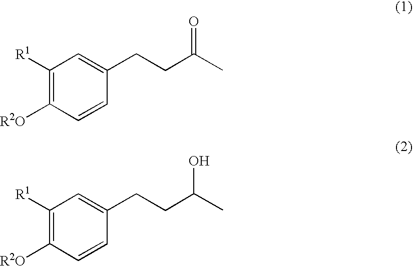
wherein R¹ is H, OH, or OCH₃, R² is H, COR³, or a sugar group, and R³ is a C₂-C₂₀ alkyl group; and
(ii) an effective taste-masking amount of one or more extract selected from the group consisting of extracts of oolong tea, puerh tea, roasted adlay tea, eucommia leaves, jasmine flower, banzakuro (guava fruit), barley, mate tea (Paraguay tea), rooibos tea, tinpi (matured peal of Citrus unshiu), juemingzi, sanzashi (Crataegus), soybean germ, ginger, ginseng, kouki tea, and yucca.

2. The composition of claim 1, wherein said one or more extract is selected from the group consisting of extracts of oolong tea, puerh tea, roasted adlay tea, eucommia leaves, and jasmine flower.

3. The composition of claim 1, wherein the compound of formula (1) or formula (2) is selected from the group consisting of raspberry ketone, zingerone and BK-180.

4. The composition of claim 1, wherein the ratio by weight of component (i) to component (ii) ranges from about 10:1 to about 1:20.

5. The composition of claim 1, in the form of a food, beverage, dietary supplement, medical food, pet food, cosmetic, or topical application patch.

6. A method of reducing or controlling weight and/or fat comprising administering an effective amount of the composition according to claim 1 to a mammal to reduce or control weight and/or fat.

7. A composition comprising:
(i) one or both of a compound of formula (1) or formula (2) 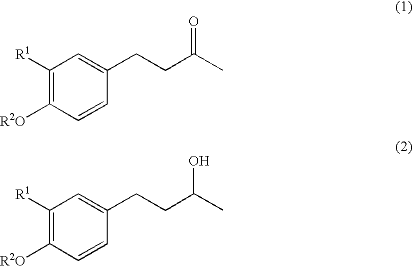
wherein R¹ is H, OH, or OCH₃, R² is H, COR³, or a sugar group, and R³ is a C₂-C₂₀ alkyl group; and
(ii) an effective taste-masking amount of one or more members selected from the group consisting of malthol, ethyl maltol, furaneol, furaneol derivatives, vanillin, and ethyl vanillin.

8. The composition of claim 7, wherein the compound of formula (1) or formula (2) is selected from the group consisting of raspberry ketone, zingerone, or BK-180.

9. The composition of claim 7, wherein the ratio by weight of component (i) to component (ii) ranges from about 10:1 to about 1:20.

10. The composition of claim 7, in the form of a food, beverage, dietary supplement, medical food, pet food, cosmetic, or topical application patch.

11. A method of reducing or controlling weight and/or fat comprising administering an effective amount of a composition according to claim 7 to a mammal to reduce or control weight and/or fat.

12. A clathrate comprising a compound represented by formula (1) or formula (2) 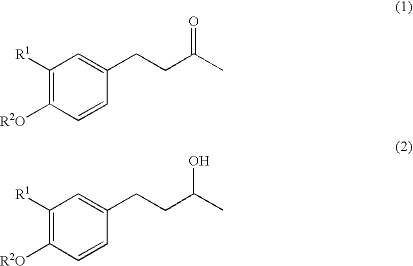
wherein R¹ is H, OH, or OCH₃, R² is H, COR³, or a sugar group, and R³ is a C₂-C₂₀ alkyl group, included in a cyclodextrin.

13. The clathrate of claim 12, wherein said cyclodextrin is a β-cyclodextrin.

14. The clathrate of claim 12, wherein said compound of formula (1) or formula (2) is selected from the group consisting of raspberry ketone, zingerone, and BK-180.

15. A composition comprising the clathrate of claim 12, in the form of a food, beverage, dietary supplement, medical food, pet food, cosmetic, bath salt, or topical application patch.

16. A method of reducing or controlling weight and/or fat comprising administering an effective amount of the clathrate of claim 12 to a mammal to reduce or control weight and/or fat.
